(19) United States
(12) Patent Application Publication
(10) Pub. No.: US 2026/0151333 A1
(43) Pub. Date: Jun. 4, 2026

(54) GASTRORETENTIVE SYSTEMS AND METHODS OF MAKING AND USING SAME

(71) Applicant: Merck Sharp & Dohme LLC, Rahway, NJ (US)

(72) Inventors: Sahab Babaee, Jersey City, NJ (US); John B. Cline, Monroe, NJ (US); Michael Galluppi, Bridgewater, NJ (US); Guangli Hu, Summit, NJ (US); Christopher Granelli, Chatham, NJ (US); Edward Yeung, Whitestone, NY (US)

(73) Assignee: Merck Sharpe & Dohme LLC, Rahway, NJ (US)

(21) Appl. No.: 19/123,982

(22) PCT Filed: Oct. 26, 2023
(86) PCT No.: PCT/US2023/077826

§ 371 Date: Apr. 24, 2025

Related U.S. Application Data
(60) Provisional application No. 63/421,274, filed on Nov. 1, 2022.

(51) Int. Cl. A61K 9/00

(52) U.S. Cl. CPC A61K 9/0065; A61M 2210/1053

(57) ABSTRACT

In some examples, a gastroretentive system includes a dissolvable outer capsule, a body disposed within the outer capsule, at least one expandable and collapsible member coupled to the body and configured to be disposed within the outer capsule in a collapsed condition when the outer capsule is present and to transition to an expanded condition without the outer capsule, the expanded condition being too large to pass through a human pylorus, wherein at least one of the body and the at least one expandable and collapsible member includes a drug product.

### FIELD OF THE DISCLOSURE

The present disclosure relates generally to drug delivery systems. More specifically, the present disclosure relates to gastroretentive systems for drug delivery.

### BACKGROUND OF THE DISCLOSURE

Capsules and tablets are used to orally deliver medications to patients in an efficient and painless manner. Tablets are generally composed of a medication, while capsules are shells that encapsulate a medication. Capsules are small enough to be swallowed and generally come in two types. Soft capsules (or “soft-gels”) are one-piece capsules that are hermetically sealed and typically contain liquids and/or semisolid fills that are introduced with the production of the capsule. Hard capsules are typically formed of gelatin and produced prior to filing the active ingredient by a manufacturer. A standard gelatin hard capsule may dissolve in the stomach in approximately twenty to thirty minutes. Standard tablets and capsules are, however, not suitable for drug delivery over days, weeks or months.

Thus, there exists a need for devices that improve upon and advance the methods of delivering drugs and medicaments to patients in a safe, comfortable and controlled manner over longer periods of time.

### SUMMARY OF THE DISCLOSURE

In some embodiments, a gastroretentive system includes a dissolvable outer capsule, a body disposed within the outer capsule, at least one expandable and collapsible member coupled to the body and configured to be disposed within the outer capsule in a collapsed condition when the outer capsule is present and to transition to an expanded condition without the outer capsule, the expanded condition being too large to pass through a human pylorus, wherein at least one of the body and the at least one expandable and collapsible member includes a drug product.

In some embodiments, a gastroretentive system includes a dissolvable outer capsule, a body disposed within the outer capsule, and a plurality of springs coupled to the body, each of the plurality of springs having a compressed condition when disposed within the outer capsule and a relaxed condition when removed from the outer capsule, the plurality of springs, in relaxed conditions, being configured and arranged to prevent the body from passing through a human pylorus, wherein at least one of the body and plurality of springs includes a drug product.

In some embodiments, a method of delivering a drug product includes providing a gastroretentive system comprising a dissolvable outer capsule, a body disposed within the outer capsule, and a plurality of springs coupled to the body, each of the plurality of springs having a compressed condition when disposed within the outer capsule and a relaxed condition when removed from the outer capsule, wherein at least one of the body and plurality of springs comprises a drug product, swallowing the outer capsule, dissolving the outer capsule, and permitting the plurality of springs to transition to relaxed conditions to impede passage of the body through a human pylorus.

### BRIEF DESCRIPTION OF THE DISCLOSURE

Various embodiments of the presently disclosed gastroretentive systems are disclosed herein with reference to the drawings, wherein:

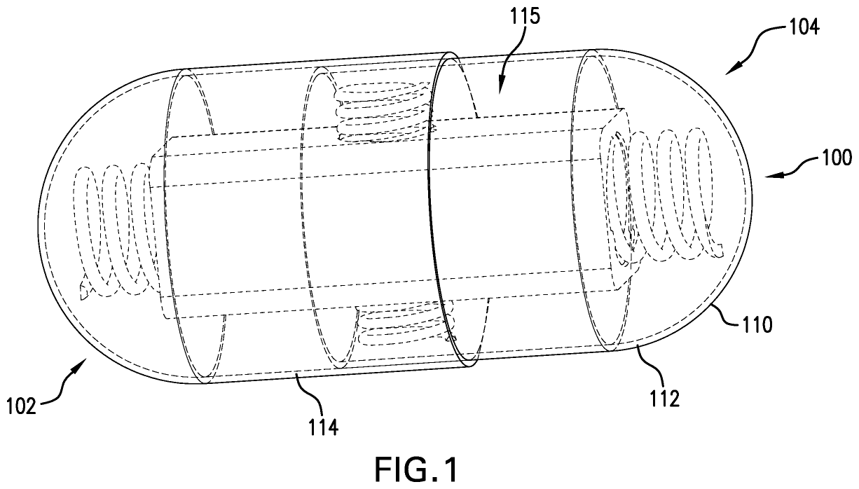

FIG. 1 is a schematic perspective view of a gastroretentive system according to one embodiment;

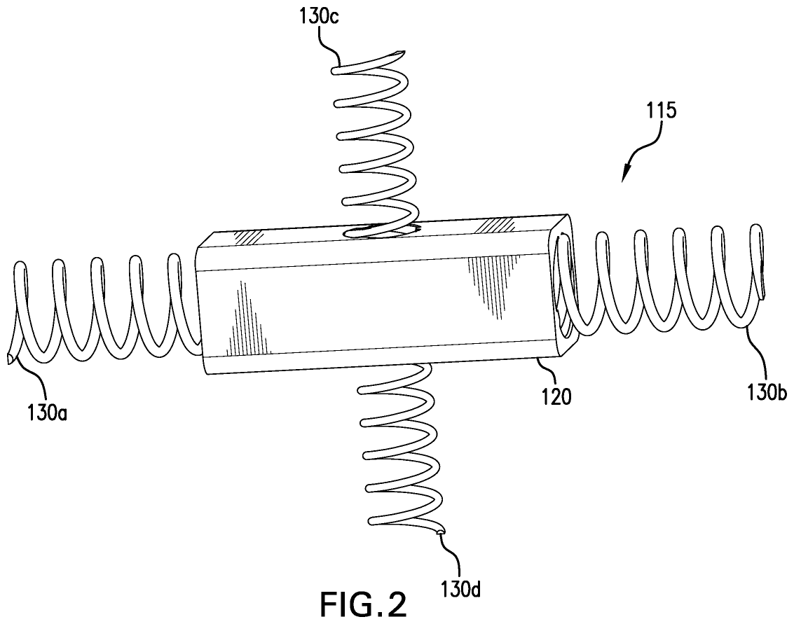

FIG. 2 is a schematic perspective view of a retentive subunit according to one embodiment;

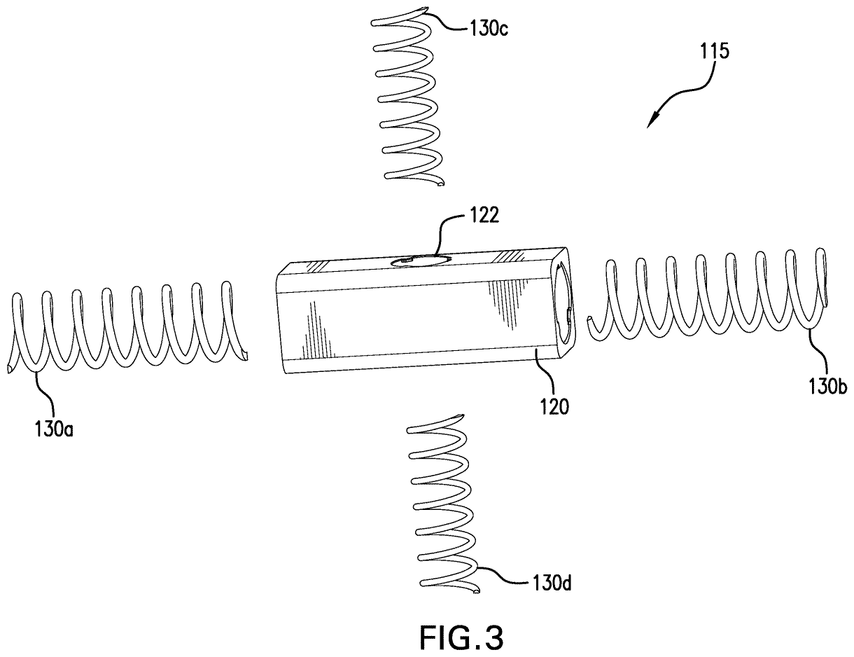

FIG. 3 is a schematic exploded view of the retentive subunit of FIG. 2;

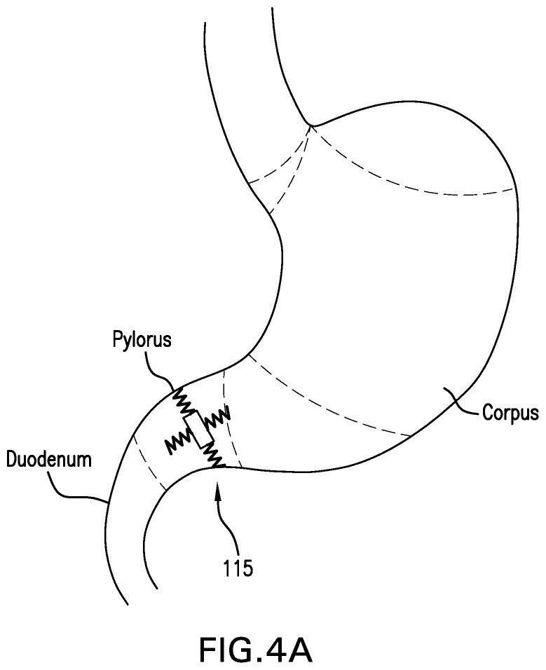

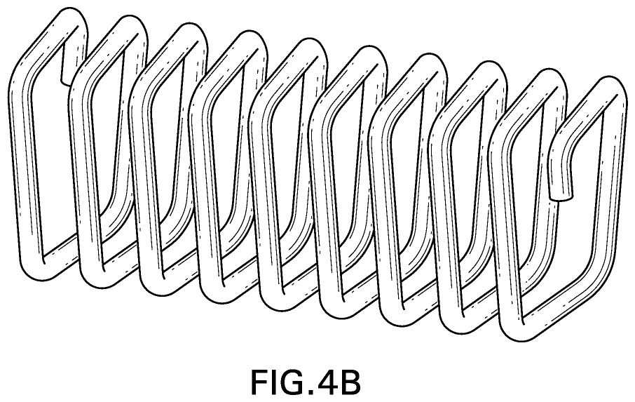

FIG. 4A is a schematic illustration of a retentive subunit disposed adjacent the pylorus;

FIG. 4B is a schematic perspective view of another example of a spring for use with a retentive subunit;

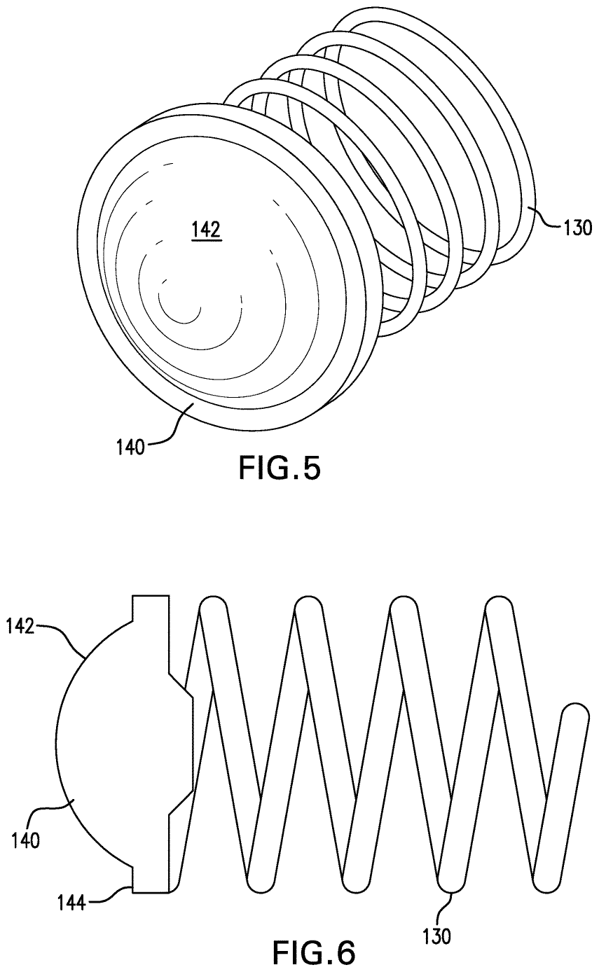

FIGS. 5-6 are schematic perspective and cross-sectional views of an atraumatic blunt nose on the distal end of a spring according to one example;

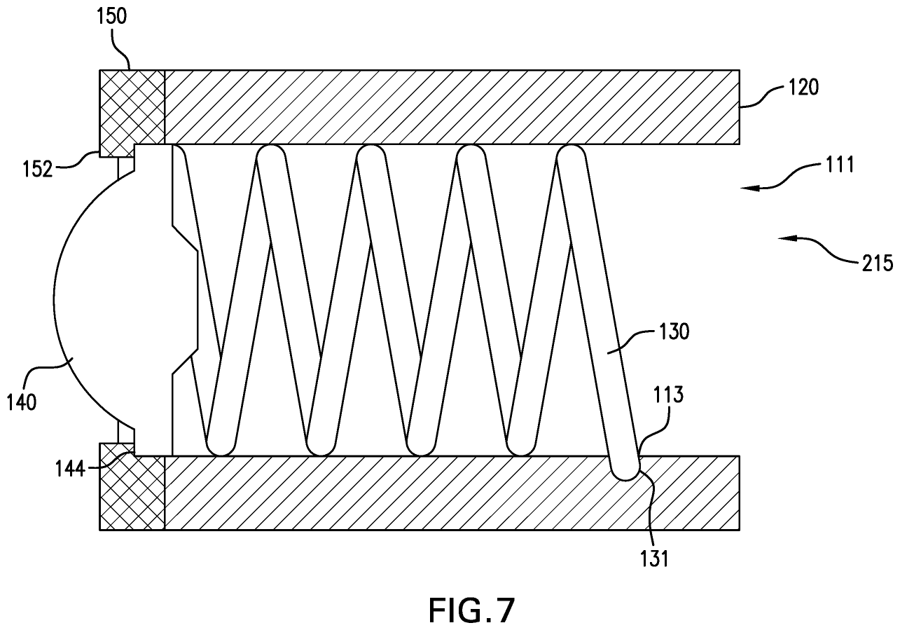

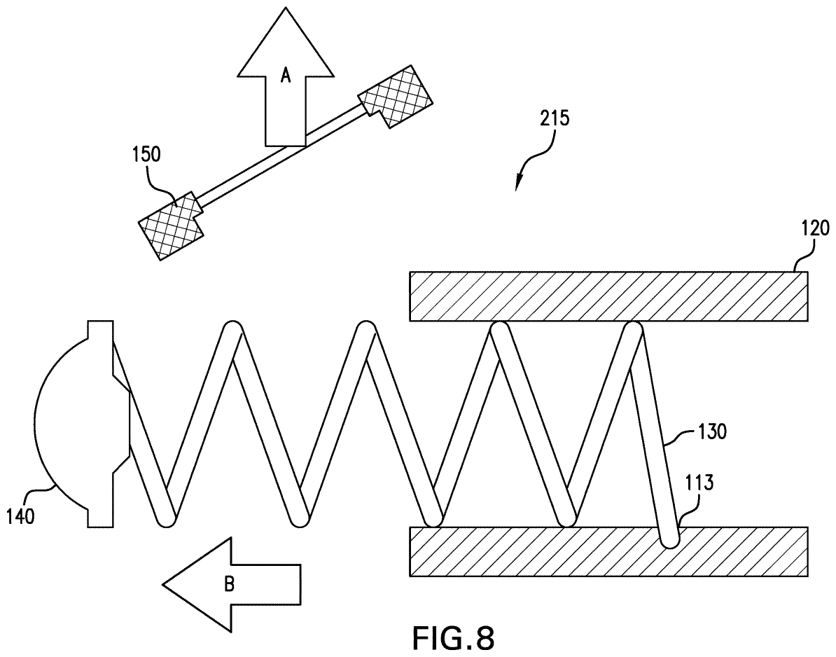

FIGS. 7-8 are schematic cross-sectional views showing the transition of a spring from a compressed condition to a relaxed condition;

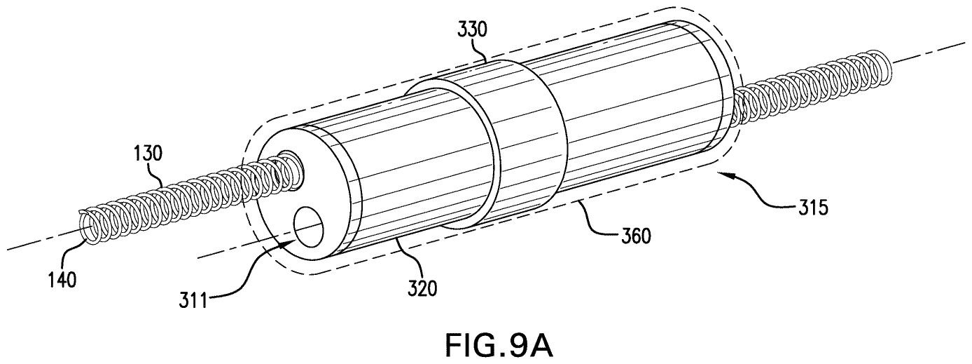

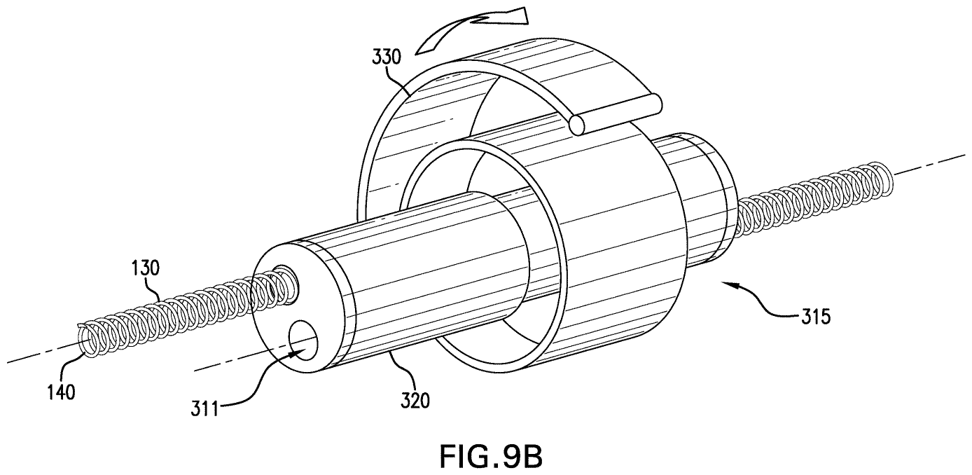

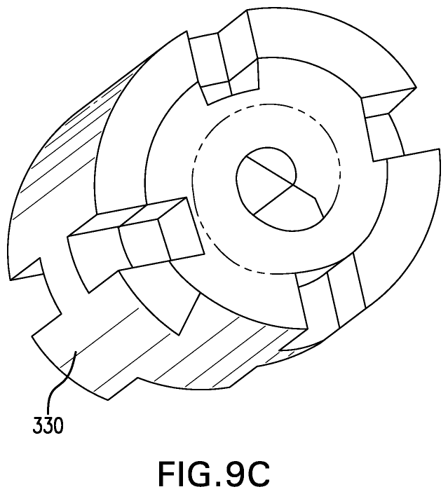

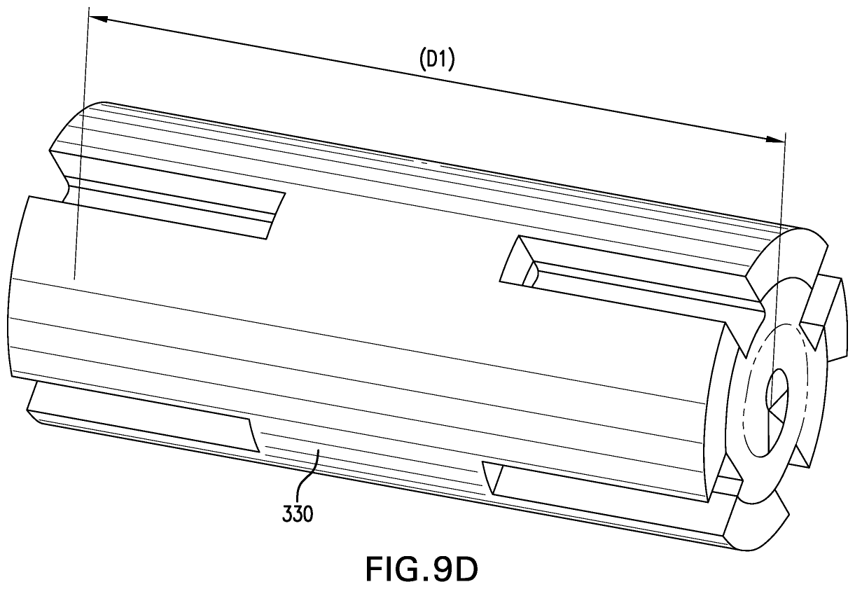

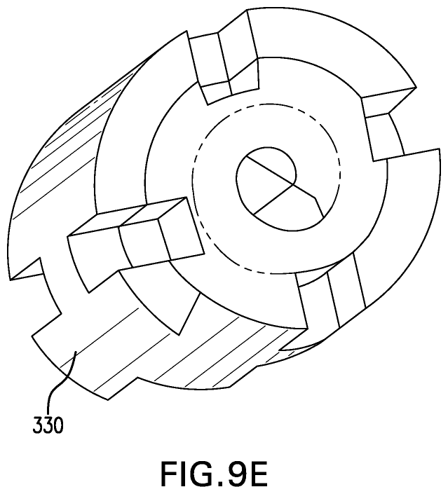

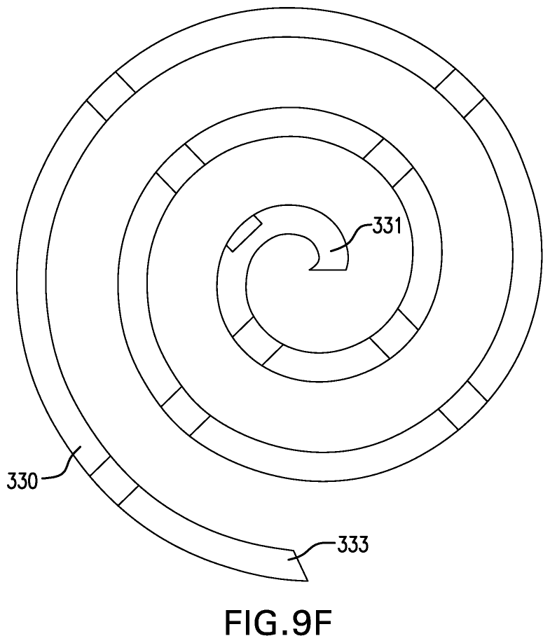

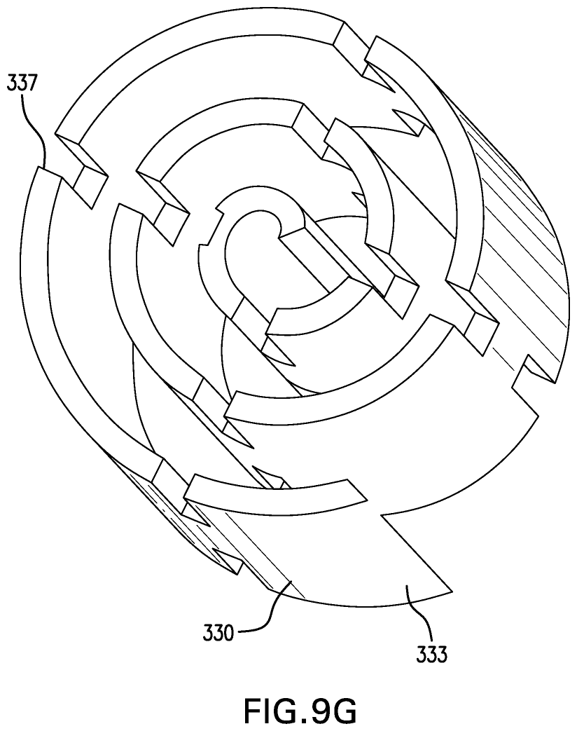

FIGS. 9A-B are schematic perspective views of another retentive subunit having a winding strap;

FIGS. 9C-G are schematic views of one example of a strap in wound and unwound conditions; and

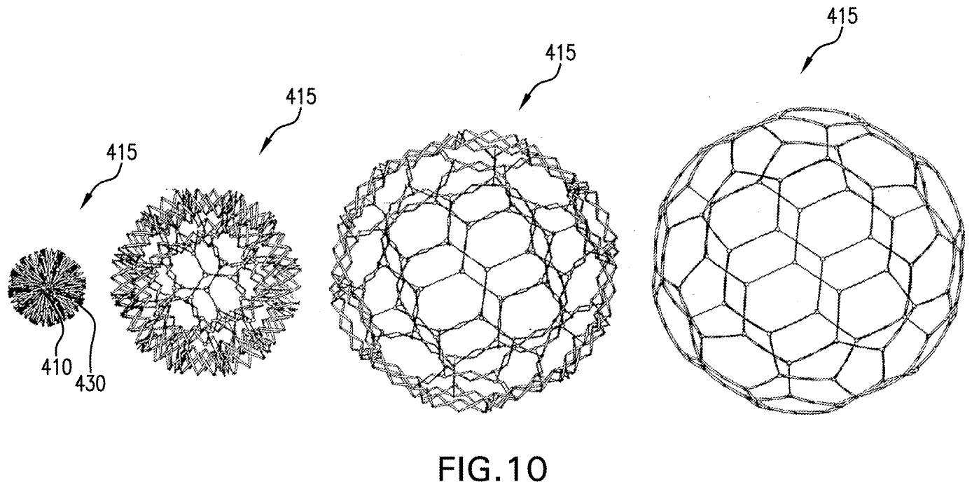

FIG. 10 are schematic perspective views of another example of a spherical retentive subunit.

Various embodiments will now be described with reference to the appended drawings. It is to be appreciated that these drawings depict only some embodiments of the disclosure and are therefore not to be considered limiting of its scope.

### DETAILED DESCRIPTION

Despite the various improvements that have been made to tablets and capsules, conventional methods of drug delivery suffer from some shortcomings as discussed above. Therefore, there is a need for further improvements to the devices and methods used to deliver medication. Among other advantages, the present disclosure may address one or more of these needs.

As used herein, the terms “medicament,” “substance,” “drug product,” and drug are used interchangeably, and it will be understood that the gastroretentive systems of the present disclosure may be used to deliver any biologic, therapeutic, drug, drug product, chemical composition, saline and/or other substances to heal or treat a disease or condition.

FIG. 1 shows one example of a gastroretentive system 100, which extends between a first end 102 and a second end 104. In this example, gastroretentive system 100 includes a dissolvable outer capsule 110 in the form of a gelatin-based hard capsule. The outer capsule 110 includes two parts 112,114 coupleable together to define an interior. In some examples, outer capsules 110 may be a “00” or “000” sized capsule having a volume of 0.91 ml or 1.37 ml. Outer capsule 110 may be configured to dissolve within the stomach within 2-30 minutes. In at least some examples, outer capsule 110 is configured to dissolve within 2-5 minutes of swallowing to ensure that the delivery system works as intended. It will be understood that other types of capsules (e.g., softgels, one-piece, etc.) are also possible. As shown in FIG. 1, a retentive subunit 115 is housed within the interior of outer capsule 110 and will be exposed to the environment when the outer capsule 110 is removed or dissolved. Retentive subunit 115 is shown collapsed within outer capsule 110 of FIG. 1, and it will be deployed once the encapsulating ingestible capsule 110 dissolves.

Turning to FIG. 2, retentive subunit 115 is shown by itself without the outer capsule 110, and generally includes a body 120 and one or more springs 130. In the example shown, body 120 is shown as being cuboid (or as a rectangular prism), but other shapes of the body 120 are possible including cubed, spheroid, etc. In at least some examples, body 120 is formed of a polymer, gelatin or other suitable biocompatible medium that is impregnated, coated or otherwise formed to deliver a drug product for sustained release.

Retentive subunit 115 may further include one or more springs 130 coupled to, or supporting, body 120, each spring having a compressed condition (for delivery) and a relaxed condition (for fixation). In the example shown, springs 130 include four springs 130a-d coupled to faces of the body 120. Two of the springs 130a,b, form a first set of springs that extend along a horizontal axis, while another two of the springs 130c,d form a second set of springs that extend along a vertical axis. It will be understood that any of springs 130a-d may be disposed in different orientations as needed. Springs 130 may be formed of a metal (e.g., nitinol), polymer or other suitable biocompatible material that has a suitable elastic deformation profile that allows it to transition between a compressed condition and a relaxed condition. In at least some examples, in the relaxed condition, the springs are sized, alone or collectively, to prevent retentive subunit 115 from passing through a human pylorus to allow for prolonged drug delivery within the stomach. That is, upon the dissolution of the capsule, the springs 130 will return to their uncompressed free length. For example, the first set of springs may collectively form a first length of greater than, or equal to, 25 mm in the relaxed condition, and the second set of springs may collectively form a second length of greater than, or equal to, 25 mm in the relaxed condition. The length of each spring 130 may be varied as needed. For example, all of the springs may be formed of a same length or different lengths, depending on the size and/or shape of body 120. Additionally, the number of helical coils or revolutions formed by the springs may vary as well as the spring constant. The shape of springs 130 may also be different. For example, the spring may have a circular cross-section as shown or other cross-sectional shape (e.g., a trapezoidal-shaped cross-section, a rectangular-shaped cross-section, or a square-shaped cross-section as shown in FIG. 4B). In at least some examples, one or more of springs 130 may be impregnated, coated or otherwise configured to deliver a drug product for sustained release in addition to the body 120, or instead of it. FIG. 3 shows an exploded view of the retentive subunit 115, which better shows bores or seats 122 of body 120 which will receive the springs 130a-d.

By allowing retentive subunit 115 to transition from a collapsed state to an expanded state, retentive subunit 115 is prevented from passing through the pylorus at any angle and may indwell in, or adjacent, the pylorus for a predetermined period of time until it breaks down into its components. (See, FIG. 4A). In at least some examples, retentive subunit 115 will remain within the stomach for a period of one week to three months. Body 120 may be configured to dissolve gradually within the patient's body. Alternatively, joints or connections between body 120 and springs 130 may deteriorate so that the components are decoupled from one another and may pass through the pylorus and the intestines. In at least some examples, all of the components of subunit 115 are dissolvable. Alternatively, some of the components are dissolvable and others are durable and will be passed through the patient's body.

It may be advantageous for the spring 130 to have a blunt or non-piercing exposed tip to avoid injury or irritation to surrounding tissue. In one example, an atraumatic blunt nose 140 having a bulbous or hemispherical surface 142 is integrated with, or coupled to the distal ends of some or all of the springs as shown in FIGS. 5-6. The atraumatic blunt nose 140 may be formed of a metal or polymer, and may be made of the same or different material as spring 130. In at least some examples, atraumatic blunt nose 140 itself is impregnated, coated or otherwise configured to deliver a drug product. Atraumatic blunt nose 140 may be fitted to each spring as a separate component or may be integrally molded as part of the spring. In some examples, atraumatic blunt nose 140 may be used to compress the spring 130 to a restricted length until the retentive subunit is ready for deployment. To achieve this, nose 140 may also include a brim 144 to help keep the spring in the compressed condition (FIG. 6).

As shown in FIG. 7, a second example of a retentive subunit 215 includes a body 120 defining a lumen 111 for receiving a majority of a compressed spring 130, and an inner undercut or notch 113 for receiving and fixing one end (e.g., a latching pin 131) of spring 130. Notch 113 will keep spring 130 fixed in place and prevent the release of the spring until the body 120 begins to dissolve shortly after dissolution of the capsule. Other examples of coupling the spring 130 to body 120 are also contemplated including swaged holes or bonding via adhesives or other means.

A retention cap 150 may be snap-fit, adhesively bonded, integrally formed with, or otherwise coupled to body 120. Retention cap 150 may be frangibly or removably coupled to body 120 and configured to break off, dissolve or otherwise dissociate from body 120 to release the compressed springs. Retention cap 150 may include discrete tabs or a continuous circumferential lip 152 with a decreased diameter (i.e., a diameter that is smaller than the diameter of brim 144) to retain atraumatic blunt nose 140 and keep spring 130 from extending to its relaxed condition. When retention cap 150 is decoupled or removed as shown by arrow “A” in FIG. 8, spring 130 will return to its longer, relaxed condition in the direction of arrow “B”.

After, or while, delivering the medication over the course of weeks or months, body 120 of subunit 215 may begin to slowly dissolve, and will eventually release the springs for discharge through the gastrointestinal tract.

FIGS. 9A-B show a similar concept with some minor variations. Specifically, retentive subunit 315 may be disposed with a dissolvable capsule as previously disclosed. In this example, retentive subunit 315 includes a generally cylindrical body 320 having bores 311 for accepting one or more opposable springs 130, each spring having an atraumatic blunt nose 140. In addition to the shape of body 320, a difference in this example is the use of an expanding strap 330, coupled to body 320. Strap 330 may extend around the circumference of body 320 and may have an expanded condition and a collapsed condition. In one example, strap 330 may be formed of a biocompatible plastically deformable material (e.g., metal, polymer, etc.) and/or a shape-memory material, and may have a coiled configuration that is capable of transitioning between a wound condition (FIG. 9A) and an unwound condition (FIG. 9B). In the unwound condition, the strap may have a diameter that is equal to, or greater, than 25 mm. In the wound condition, the strap may have a reduced diameter to fit within a dissolvable capsule. As shown in FIG. 9A, in one embodiment, the clockwork spring may be held in its tightened state by a rapidly-dissolving sleeve 360 of film that begins to dissolve once the surrounding capsule dissolves.

FIGS. 9C-G illustrates several views of strap 330 in both the collapsed and expanded conditions including a cross-sectional view (FIG. 9F) for appreciating the fixed end 331 of strap 330 that may couple to body 320, and free end 333 that will uncoil or unwind. In some examples, strap 330 may be in the form of a clockwork spring that is expandable in the transverse plane. The clockwork spring may be affixed to the outer surface of body 320 at fixed end 331 in its tightly wound state. In one embodiment, the clockwork spring may be retained in place by bonding or swaging it in place on the body, or by a snap fit for retaining it in place on body 320. The clockwork spring may take up minimal volume while wrapped around the body but may expand to the desired diameter upon dissolution of the rapidly-dissolving sleeve. Strap 330 itself may be made of a dissolvable material and could have sufficient notches 337 (best shown in FIG. 9G) to allow for segments of it to be expelled over time as the smaller section dissolves. Strap 330 may be formed as a spiral that is then tightly wound, or as a stiff flat ribbon that can be rolled tightly.

It is to be understood that the embodiments described herein are merely illustrative of the principles and applications of the present disclosure. Other configuration, shapes and elements may be used to create at least one expandable and collapsible member and/or a body. For example, the expandable and collapsible member(s) may include one or more, or a combination of, coil springs, clockwork torsion springs, leaf springs, pH or temperature-activated expanding foams and the like. Additionally, the body may include cylindrical, cuboid, or even spherical bodies, so long as they are capable of fitting within a capsule for delivery in a compressed condition. The expandable and collapsible members and the body may be arranged in many variations and may be linked together with connectors. For example, radial expansion-retraction truss structures (known as “hoberman spheres”) may be used to construct retentive subunits 415 similar to those shown in FIG. 10. Other suitable shapes are disclosed in U.S. Pat. No. 5,025,031 to Hoberman, which is hereby incorporated by reference in its entirety. Moreover, the number, positioning and arrangement of body and springs of retentive subunits may be varied. Additionally, the shape, dimensions and arrangement of the body may also be varied, and the type and amount of the drug product may be chosen based on the retentive subunit. Moreover, certain components are optional, and the disclosure contemplates various configurations and combinations of the elements disclosed herein. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A gastroretentive system comprising:
an outer capsule capable of dissolving in the stomach;
a body disposed within the outer capsule; and
at least one expandable and collapsible member coupled to the body and configured to be disposed within the outer capsule in a collapsed condition when the outer capsule is present and to transition to an expanded condition without the outer capsule, the expanded condition being impassable through a human pylorus, wherein at least one of the body and the at least one expandable and collapsible member comprises a drug product.

2. The gastroretentive system of claim 1, wherein the body is substantially in a shape of a cuboid.

3. The gastroretentive system of claim 1, wherein the at least one expandable and collapsible member comprises at least one spring having a compressed condition and a relaxed condition.

4. The gastroretentive system of claim 3, wherein the at least one spring comprises multiple springs.

5. The gastroretentive system of claim 3, wherein the at least one spring comprises a first set of springs extending along a first axis, and a second set of springs extending along a second axis, the first axis and the second axis being orthogonal.

6. The gastroretentive system of claim 5, wherein the first set of springs collectively form a first length of at least 25 mm in the relaxed condition, and the second set of springs collectively form a second length of at least 25 mm in the relaxed condition.

7. The gastroretentive system of claim 3, wherein the at least one spring comprises an atraumatic nose disposed on a distal end thereof.

8. The gastroretentive system of claim 7, wherein the atraumatic nose comprises a drug product.

9. The gastroretentive system of claim 3, further comprising a removable retention cap disposed adjacent the body and capable of maintaining the at least one spring in the compressed condition.

10. The gastroretentive system of claim 1, wherein the outer capsule is configured to dissolve in a period of less than one hour.

11. The gastroretentive system of claim 3, wherein a majority of the at least one spring is disposed within the body in the compressed condition.

12. The gastroretentive system of claim 3, wherein the at least one spring is fully disposed within the body in the compressed condition.

13. The gastroretentive system of claim 1, wherein the at least one expandable and collapsible member further comprises at least one winding strap coupled to the body and having a wound condition and an unwound condition.

14. The gastroretentive system of claim 1, wherein the body is configured and arranged to break down after more than one week and to decouple from the at least one expandable and collapsible member.

15. A gastroretentive system comprising:
a dissolvable outer capsule;
a body disposed within the outer capsule; and
a plurality of springs coupled to the body, each of the plurality of springs having a compressed condition when disposed within the outer capsule and a relaxed condition when removed from the outer capsule, the plurality of springs, in relaxed conditions, being configured and arranged to prevent the body from passing through a human pylorus, wherein at least one of the body and plurality of springs comprises a drug product.

16. The gastroretentive system of claim 15, wherein the plurality of springs comprises a first set of springs extending along a first axis, and a second set of springs extending along a second axis, the first axis and the second axis being orthogonal.

17. The gastroretentive system of claim 16, wherein the first set of springs collectively form a first length of at least 25 mm in the relaxed condition and the second set of springs collectively form a second length of at least 25 mm in the relaxed condition.

18. A method of delivering a drug product, comprising:
providing a gastroretentive system including a dissolvable outer capsule, a body disposed within the outer capsule, and a plurality of springs coupled to the body, each of the plurality of springs having a compressed condition when disposed within the outer capsule and a relaxed condition when removed from the outer capsule, wherein at least one of the body and plurality of springs comprises a drug product;
swallowing the outer capsule;
dissolving the outer capsule; and
permitting the plurality of springs to transition to relaxed conditions to impede passage of the body through a human pylorus.

19. The method of claim 18, further comprising the step of decoupling the body from the plurality of springs after more than one week and allowing any remaining components to pass through the human pylorus.

20. The method of claim 18, further comprising the step of releasing the drug product into a human stomach over a period of at least one week.
